(19) United States
(12) Patent Application Publication
(10) Pub. No.: US 2014/0350117 A1
(43) Pub. Date: Nov. 27, 2014

(54) Topical Scar Treatment Composition and Method of Using Same

(71) Applicant: Fabiana Campanati Banov, Sugar Land, TX (US)

(72) Inventor: Fabiana Campanati Banov, Sugar Land, TX (US)

(73) Assignee: PROFESSIONAL COMPOUNDING CENTERS OF AMERICA, Houston, TX (US)

(21) Appl. No.: 13/900,379

(22) Filed: May 22, 2013

(51) Int. Cl. A61K 31/138

(52) U.S. Cl. CPC A61K 31/138
USPC 514/648

(57) ABSTRACT

The present disclosure describes a topical scar treatment composition that may include tamoxifen citrate in a concentration of about 0.01% by weight to about 1% by weight, with about 0.1% by weight being preferred, and an anhydrous silicone base for enhancing skin permeation and scar removing effects, in a concentration of about 10% by weight to about 100% by weight, with about 100% being preferred being preferred. The small concentration of tamoxifen citrate may prevent dangerous side effects and may be successful in treating abnormal scars such as keloids and hypertrophic scars.

### CROSS-REFERENCE TO RELATED APPLICATIONS

N/A

### BACKGROUND

1. Field of the Disclosure

The present disclosure relates to pharmaceutical compositions, and more specifically to a topical scar treatment composition for treating excessive scarring that may result in hypertrophic scars and keloids.

2. Background Information

When a wound heals, a scar takes its place. A scar is growth of collagen beneath the skin that is formed as the result of wound healing; therefore, every cut or injury to the skin heals to form a scar. Proper wound healing may result in an invisible scar. Early in the process, scars are red or dark and raised, but may become paler and flatter over time. While a visible scar may be the necessary and inevitable end to the healing process, the results vary with the individual, the type of injury, and time, among others. For example, when scars are over about two months old to about two years old, they tend to become harder to treat, and after two years a surgery may be necessary to remove them, which may even result in new scars appearing after the removal surgery.

Excessive scarring results from an imbalance in the anabolic and catabolic wound healing processes. In the formation of an abnormal scar, more collagen is produced than is degraded. As a result, the scar grows larger than is required for wound healing, with an over-production of cells, collagen, and proteoglycan. Scars resulting from excessive scarring or the abnormalities in wound healing may include fibrosis, fibromatosis, keloidosis, adhesions (e.g. surgical adhesions), hypertrophic scars, fibrocystic conditions, and joint stiffness. For example, keloids grow in all directions, become elevated above the skin, and remain hyperemic. The exact mechanisms of excessive scarring are poorly understood, but it is believed that common mechanisms underlie the formation of both keloids and hypertrophic scars. Evidence suggests that increased transforming growth factor β (TGF-β) expression plays a role in excessive scarring, due to promoting extracellular matrix production, and because of being produced at elevated levels by keloid fibroblasts and hypertrophic scars. Abnormal scars or abnormalities in wound healing can also be categorized into various conditions based on the type of tissue in which a wound occurs. Abnormal scar formation in skin may lead to, for example, keloid, hypertrophic scar, contracture, or scleroderma.

As an alternative keloid and hypertrophic scar treatment, some researchers have proposed using the breast cancer drug tamoxifen, which has shown to inhibit keloid fibroblast proliferation and decreases collagen production. Apparently, tamoxifen affects this inhibition by downregulating TGF-β expression. However, tamoxifen is currently available mainly for oral administration, involving delivery of high doses of this drug to the body (about 10 to about 20 mg/day), causing health risks and significant unwanted side effects. These effects include the increased risk of endometrial cancer, endometrial hyperplasia and polyps, deep vein thrombosis and pulmonary embolism, changes in liver enzyme levels, and ocular disturbances, including cataracts. Additionally, patients treated with oral tamoxifen citrate have reported having hot flashes, vaginal discharge, depression, amenorrhea, and nausea. Locally administered tamoxifen, which might pose fewer risks, would eliminate first-pass liver metabolism, which changes tamoxifen into its active metabolites.

Accordingly, improved methods for treating excessive scarring, such as keloids and hypertrophic scars, overcoming problems of the prior art, may be needed.

### SUMMARY

The present disclosure describes a topical scar treatment composition that may include tamoxifen citrate in concentrations of about 0.01% by weight to about 1% by weight, with about 0.1% by weight being preferred. The tamoxifen citrate within topical scar treatment composition may be combined with a pharmaceutically acceptable vehicle, being anhydrous silicone a preferred vehicle. Anhydrous silicone base may be included in concentrations of about 10% by weight to about 100% by weight, with about 100% being preferred. The topical scar treatment composition may be administered in any suitable topical dosage form.

Tamoxifen citrate, an anti-estrogen drug that has generally been used for the treatment of breast cancer, when prepared into the topical scar treatment composition, may be helpful in treating abnormal proliferative healing disorders. It is believed that one of the mechanisms by which tamoxifen citrate decreases keloid fibroblast proliferation and collagen synthesis is by downregulating TGF-β, although other mechanisms may include RNA transcription alteration, cell G1 phase delay or arrest, and insulin growth factor (IGF) suppression, among others. Concentrations greater than about 20 μM (corresponding to a tamoxifen citrate concentration of about 0.01%) have a deadly effect on keloid and hypertrophic scar cells.

Generally, tamoxifen citrate is found in oral dosage form, and doses may be as high as 20 mg/day. These high doses may cause considerable side effects, including flushing, skin rash, nausea, vomiting, weight gain, myelosuppression, hepatotoxicity, and neuromuscular pain. Thus, the low concentration (about 0.1% or 1 mg/g) of tamoxifen citrate within topical scar treatment composition, may be high enough to kill keloid and hypertrophic scar cells, but low enough to prevent considerable side effects.

According to an embodiment, topical scar treatment composition may include permeation enhancers, such as an anhydrous silicone base, which may improve the penetration of tamoxifen citrate in skin as well as provide moisture and healing properties. Ingredients within anhydrous silicone base may include Amazonian oils, such as pracaxi oil and seje oil, which may be rich sources of essential fatty acids, behenic acid, oleic acid, and in some instances, lauric acid. The supply of essential fatty acids and antioxidant molecules may restore cutaneous permeability. The supply of essential fatty acids and antioxidant molecules may also contribute to the control of the imperceptible water loss and maintain moisture of the skin.

Numerous other aspects, features and benefits of the present disclosure may be made apparent from the following detailed description taken together with the drawing FIGURES.

### BRIEF DESCRIPTION OF THE DRAWINGS

The present disclosure can be better understood by referring to the following FIGURES. The components in the figures are not necessarily to scale, emphasis instead being placed upon illustrating the principles of the disclosure. In the FIGURES, reference numerals designate corresponding parts throughout the different views.

FIG. 1 shows effects of tamoxifen citrate on TGF-β, according to an embodiment.

### DETAILED DESCRIPTION

The present disclosure is here described in detail with reference to embodiments illustrated in the drawings, which form a part here. Other embodiments may be used and/or other changes may be made without departing from the spirit or scope of the present disclosure. The illustrative embodiments described in the detailed description are not meant to be limiting of the subject matter presented here.

### DEFINITIONS

As used here, the following terms may have the following definitions:

“Treating” and “Treatment” refer to reduction in severity and/or frequency of symptoms, elimination of symptoms and/or underlying cause, prevention of the occurrence of symptoms and/or their underlying cause, and improvement or remediation of damage.

“Abnormal scar” refers to the over-expression of collagen at a wound site or in a scar, leaving an unaesthetic mark. Abnormal scars may include keloids and hypertrophic scars, among others.

“Excessive scarring” refers to a process that may result from an imbalance in the anabolic and catabolic wound healing processes, causing overgrowth of dense fibrous tissue and which may bring about one or more abnormal scars. Excessive scarring may be characterized by overproduction of cells, collagen, and/or proteoglycan.

“Keloids” refers to abnormal scars resulting from benign fibrous growths that occur after trauma or wounding of the skin, which may extend beyond original areas of skin injury, and which tend to remain elevated.

“Hypertrophic scars” refers to abnormal scars in which dense fibrous tissue does not extend beyond borders of an original wound or incision, and which tend to be wider than necessary for normal wound healing to occur. Histologically, hypertrophic scars have more organized collagen fibers than keloids, and scant mucoid matrix. Hypertrophic scars are also characterized by randomly distributed tissue bundles including uniaxially oriented extracellular matrix and cells, and tend to occur when a trauma or injury has been inflicted to deep dermis.

“Silicone” refers to polymeric organic silicon compounds obtained as oils.

“Antiestrogenic compound” refers to an agent that blocks an activity of estrogen, and which may act to competitively or non-competitively inhibit the binding of estrogen to one of its receptors.

### DESCRIPTION OF THE DRAWINGS

The present disclosure describes a topical scar treatment composition that may include tamoxifen citrate combined with an anhydrous silicone base. Tamoxifen citrate may be used in a concentration of about 0.01% by weight to about 1% by weight, with about 0.1% by weight being preferred, a small concentration when compared to oral treatments involving tamoxifen citrate for breast cancer treatment. The small concentration may result in superior safety profile because of less or no side effects, and easier patient compliance.

Wound Healing

The nature of wound repair depends upon the tissues involved. An important aspect of wound healing is the rate at which a wound gains tensile strength. Skin exhibits tension and extensibility, this skin tension is one of the determining factors in the response to a wound and varies with age and site. Skin has multiple layers, including keratin, epidermis, and dermis, a fibrous network composed of collagen and elastin and an amorphous ground substance which includes protein polysaccharides, glycoproteins, globular proteins, salts, and water. If only the epidermis is damaged, as in most minor injuries, keratinocytes migrate from the edge of the wound and eventually cover the wound, reforming the epidermis and keratin.

If all skin layers are damaged or destroyed, new connective tissue, called granulation tissue, must first fill the wound space. Granulation tissue is formed by deposition of extracellular matrix components, for example, collagen, by fibroblasts which migrate into the wound space. The synthesis and deposition of collagen is an important event in wound healing and the rate of collagen synthesis varies in different organs.

Wound healing is commonly divided into four separate phases: coagulation, inflammation, migration and proliferation, and remodeling. Soon after a wound occurs in a subject, the wound healing process starts with a coagulation of fibrin and fibronectin to form a matrix or a clot and a gathering of platelets at the wound site. As the platelets coagulate, inflammatory cells, such as neutrophils, lymphocytes, and macrophages, are also attracted to the wound site and release factors for wound healing.

The final stage of wound healing is involved in a remodeling process which changes the deposition pattern of matrix components. As described, the initial matrix is a clot of fibrin and fibronectin resulting from homeostasis. With the proliferation and migration of fibroblasts, collagen is synthesized and deposited replacing and rearranging the initial matrix with aid from proteases. Collagen fibers gradually increase in thickness and align along the stress line of the wound. At the end of a normal scar formation, the final scar shows collagen fibers mostly parallel to the epidermis.

Thus, the wound healing process is a delicately balanced equilibrium between growth and degradation. Any aberrations in the process may tip the balance toward a pathological abnormality in wound healing or an excessive deposit of scarring tissues. For example, an excessive deposition of scar tissues in skin during a wound healing process may result in excessive scarring, thus in abnormal scars such as keloids or hypertrophic scars. In both cases, over accumulation or expression of collagen is believed to be the cause.

Transforming Growth Factor β (TGF-β)

The pathological repair process underlying keloid and hypertrophic scar formation may be mediated in part by the biological activity of transforming growth factor β (TGF-β) during wound healing. TGF-β is cytokine produced and released by platelets, fibroblasts, and endothelial, epithelial, and inflammatory cells, such as macrophages and lymphocytes, after a wound occurs. Collectively, these effects of TGF-β occurring in excess could result in the abnormal accumulation of extracellular matrix (ECM), along with fibrosis and scar formation.

TGF-β isoforms 1 and 2 are considered to be particularly involved in collagen synthesis promotion and scarring, while isoform 3 is involved in scar prevention. In wound healing, TGF-β functions by stimulating the synthesis and deposition of ECM proteins, along with decreasing metalloproteinases and increasing metalloproteinases inhibitors in the wound environment. Cumulative effects of TGF-β in excess may result in an overproduction of ECM, with scar formation and fibrosis, and a reduction in the degradation and remodeling of ECM that occurs normally during wound healing.

Keloid fibroblasts and hypertrophic scars have shown to produce increased amounts of total TGF-β compared with normal skin fibroblasts, such that TGF-β may promote excessive accumulation of ECM. Therefore, TGF-β may be the driving force behind excessive scar formation seen in abnormal scars.

Topical Scar Treatment Composition

According to an embodiment, a topical scar treatment composition may include tamoxifen citrate in concentrations of about 0.01% by weight to about 1% by weight, with about 0.1% by weight being preferred, in combination with a pharmaceutically acceptable vehicle, being anhydrous silicone a preferred vehicle. Anhydrous silicone base may be included in concentrations of about 10% by weight to about 100% by weight, with about 100% being preferred

In one embodiment, disclosed topical scar treatment composition may be administered in gel form. In other embodiments, topical scar treatment composition may be administered in other suitable topical dosage forms such as an ointment, cream, gel, emulsion (lotion), powder, oil, or similar formulation, employing suitable vehicles for each dosage form. Additionally, the topical scar treatment may include customary excipient additives, such as vegetable oils including almond oil, olive oil, peach kernel oil, groundnut oil, castor oil and the like, animal oils, DMSO, fat and fat-like substances, lanolin lipoids, phosphatides, hydrocarbons such as paraffin, petroleum jelly, waxes, detergent emulsifying agents, lecithin, alcohols, carotin, glycerol, glycerol ethers, glycols, glycol ethers, polyethylene glycol, polypropylene glycol, non-volatile fatty alcohols, acids, esters, volatile alcoholic compounds, urea, talc, cellulose derivatives, and preservatives, among others.

Tamoxifen Citrate

Tamoxifen citrate is a triphenylecthlene-derivative, nonsteroidal antiestrogen that is structurally related to clomiphene and which has been used for the treatment of breast cancer. Tamoxifen citrate occurs as a fine, white crystalline powder with a solubility of 0.5 mg/mL in water at 37° C., a pKₐ of 8.85, and is very slightly soluble in alcohol. Effects of tamoxifen citrate include altering transcriptional synthesis, decreasing cellular proliferation, arresting cells in the G1 phase of the cell cycle, and modulating the production of multiple polypeptide growth factors (TGF-α, TGF-β, epidermal growth factor, and insulin-like growth factor).

Tamoxifen citrate may be helpful in treating abnormal proliferative healing disorders. For example, tamoxifen citrate has been used to successfully treat retroperitoneal fibrosis and desmoid tumors. Furthermore, tamoxifen citrate has been demonstrated to decrease proliferation of both normal dermal and keloid fibroblasts, as well as decreasing the synthesis of collagen by keloid fibroblasts. It is believed that one of the mechanisms by which tamoxifen citrate decreases keloid fibroblast proliferation and collagen synthesis is by downregulating TGF-β, although other mechanisms may include RNA transcription alteration, cell G1 phase delay or arrest, and insulin growth factor (IGF) suppression, among others.

Clinically significant side effects of anti-estrogen agents such as tamoxifen citrate may occur when the agents displace estradiol in non-target tissues. Because tamoxifen citrate and estradiol have similar binding affinities for estrogen receptors, a competition between them for receptor binding would be approximately equal when the concentration of each compound approximates that of the other. If the tamoxifen citrate concentration exceeds the estradiol concentration, then the former may be bound preferentially to the estrogen receptors, and vice versa. Common adverse reactions to oral doses of tamoxifen citrate include flushing, skin rash, nausea, vomiting, weight gain, myelosuppression, hepatotoxicity, and neuromuscular pain.

According to an embodiment, a topical application of tamoxifen citrate at a concentration high enough to inhibit keloid and hypertrophic scar cells but also low enough to minimize side effects may be an ideal treatment for abnormal scars. To this effect, it has been shown that tamoxifen citrate concentrations between about 8 μM and about 12 μM demonstrate significant inhibition of fibroblast cells, while concentrations of about 12 μM to about 16 μM have shown a significant reduction of collagen. Other experiments have shown that tamoxifen citrate concentrations greater than about 20 μM (corresponding to a tamoxifen citrate concentration of about 0.01%) have a deadly effect on keloid and hypertrophic scar cells. Thus, a concentration of about 0.1% of tamoxifen citrate represents ten times that of the concentration that inhibits a deadly effect on keloid and hypertrophic scar cells. If the average dose were 1 g of topical scar treatment composition (for example, a gel), or less, a patient would receive only about 1 mg of tamoxifen citrate, equivalent to 1/20^{th} of the average oral dose (set at about 20 mg/day for patients on breast cancer treatment). Even if this small dose of about 1 mg of tamoxifen citrate were to be totally absorbed systemically, side effects would be considerably limited, if any.

Anhydrous Silicone Base

According to an embodiment, the topical scar treatment composition may include anhydrous silicone base that may improve the penetration of tamoxifen citrate in skin as well as provide moisture and healing properties.

Ingredients within anhydrous silicone base may include Amazonian oils such as pracaxi oil and seje oil, which may be rich sources of essential fatty acids, behenic acid, oleic acid, and in some instances, lauric acid. The supply of essential fatty acids and antioxidant molecules may restore the cutaneous permeability and the function of the skin barrier. The supply of essential fatty acids and antioxidant molecules may also contribute to the control of the imperceptible water loss and maintain moisture of the skin.

In one embodiment, anhydrous silicone base may include a viscosity modulating agent, such as a gelling agent. Concentrations of viscosity modulating agent may be determined by one skilled in the art depending on the viscosity desired in order to obtain anhydrous silicone base that may be retained in the vicinity of the area of application for a brief period of time.

Concentration of anhydrous silicone base that may be included in disclosed topical scar treatment composition may range from about 10% by weight to about 100% by weight, with about 100% being preferred.

Pracaxi Oil

In one embodiment, the anhydrous silicone base may include a natural oil from the Amazon forest, named pracaxi oil, which exhibits moisturizing properties as well as antimicrobial properties. Additionally, pracaxi oil may enhance the penetration of tamoxifen citrate in skin.

Pracaxi oil may be obtained from the seed oil of Pentaclethara Macroloba tree. Pracaxi oil may include about 20% behenic acid and about 35% oleic acid. In some cases, pracaxi oil may include more than these percentages. Behenic acid, oleic acid, and lauric acid, when used by themselves, may be irritating when applied to the skin. While having an irritating effect on the skin, behenic acid, oleic acid, and lauric acid, are also effective vehicles at delivering drugs, such as tamoxifen citrate, through the skin. As the behenic acid and oleic acid are present within pracaxi oil, the effects of the acids may be less irritating on the skin, and as such makes pracaxi oil a good skin permeation enhancer. Pracaxi oil has been widely employed for its cosmetic, therapeutic, and medicinal properties. Pracaxi oil is rich in organic acids with antioxidant, antibacterial, antiviral, antiseptic, antifungal, anti-parasitic, and anti-hemorrhagic properties; therefore, pracaxi oil is suitable oil for wound healing treatment. Pracaxi oil may also aid to lighten hyper-pigmentation caused by hormonal changes and as a direct result of wounds such as insect bites, abrasions, cuts, and acne. Additionally, pracaxi oil may improve the appearance of stretch marks.

Pracaxi oil may have a high amount of solid matter, not fatty acids, which make pracaxi oil solidify in cooler temperatures. The solid matter has gentle moisturizers and high cellular renewal properties, includes vitamin E and has essential fatty acids, which make pracaxi oil suitable oil for scar treatment.

The fatty acid composition of pracaxi oil is illustrated below in Table 1.

TABLE 1
Fatty Acid Composition of Pracaxi Oil.

| | Carbon | |
|---|---|---|
| Fatty Acidds | Atoms | Composition % |
| Lauric | 12:00 | 1.3000 |
| Myristic | 14:00 | 1.2100 |
| Palmitic | 16:00 | 2.0400 |
| Stearic | 18:00 | 2.1400 |
| Oleic | 18:10 | 44.3200 |
| Linoleic | 18:20 | 1.9600 |
| Linolenic | 18:30 | 2.3000 |
| Behenic | 22:00 | 19.6700 |
| Lignoceric | 24:00 | 14.8100 |

Concentration of pracaxi oil within anhydrous silicone base may range from about 0.5% by weight to about 15% by weight; most suitable amount may be of about 1% by weight to about 1% by weight.

Seje Oil

Seje oil may be extracted from the mesocarp of the patauá palm and generally appears as a greenish-yellow and transparent liquid, with little odor and taste, having the physical appearance and composition of fatty acids that are similar to olive oil (Olea europaea). Seje oil has high content of unsaturated fatty acids. Seje oil has a high content of oleic acid therefore seje oil may be used as skin moisturizers. The dry mesocarp of pataua palm may include about 7.4% protein and possesses an excellent amino acid composition. Seje oil also includes α-tocopherol in its composition.

The fatty acid composition of seje oil is illustrated below in Table 2.

TABLE 2
Fatty Acid composition of Seje Oil.

| | Carbon | |
|---|---|---|
| Fatty Acids | Atoms | Composition % |
| Palmitic | 16:00 | 13.2 |
| Palmitoleic | 16:10 | — |
| Stearic | 18:00 | 3.6 |
| Oleic | 18:10 | 77.7 |
| Linoleic | 18:20 | 2.7 |
| Linolenic | 18:30 | 0.6 |
| Arachidic | 20:00 | 2 |
| Unsaturated | | 81.6 |

Amount of seje oil within anhydrous silicone base may range from about 0.5% by weight to about 15% by weight; most suitable amount may be of about 1% by weight to about 5% by weight.

In further embodiments other oils such as Plukenetia Volubilis seed oil, Inaja oil, Buriti, Tucuma, Bacuri, Ucuuba, Muru-Muru, and Copaiba, may be included in anhydrous silicone base within the topical scar treatment.

Silicone

Anhydrous silicone base may include long chain silicone polymer (polysiloxanes), and silicone dioxide. Long chain silicone polymers cross link with silicone dioxide.

Silicone increases hydration of stratum corneum and therefore facilitates regulation of fibroblast production and reduction in collagen production resulting in softer and flatter scar.

Additionally, silicone within anhydrous silicone gel may protect the scarred tissue from bacterial invasion and may prevent bacteria-induced excessive collagen production in the scar tissue.

Furthermore, anhydrous silicone base may modulate the expression of growth factors, fibroblast growth factor β (FGF-β) and TGF-β.

Amount of silicone within anhydrous silicone base may range from about 10% by weight to about 95% by weight; most suitable amount may be of about 10% by weight to about 50% by weight.

Methods of Elaboration

Various methods may be used to produce disclosed topical scar treatment composition. In one embodiment, an electronic mixing system may be used employing a high shear force. Topical scar treatment composition may be mixed through number of different speed settings and times to achieve the desired particle size.

In one embodiment, in order to make topical scar treatment composition, anhydrous silicone base may be processed through an ointment mill to provide trituration, dispersion and reduce particle size of urea silicone gel. Anhydrous silicone base may be stirred under low shear conditions until a uniform formulation may be obtained. The topical scar treatment composition may be packed in suitable bottles or any suitable packaging.

Test Methods and Results

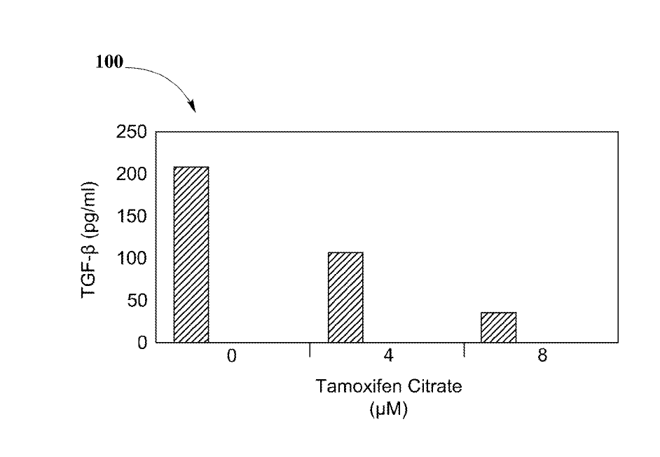

FIG. 1 shows effects of tamoxifen citrate on TGF-β 100. Accordingly, to examine the effect of tamoxifen citrate on TGF-β, a luciferase bioassay utilizing mink lung epithelial cells was employed. TGF-β is correlated with luciferase activity and reported in relative light units. As a result, tamoxifen citrate produced a dose-dependent decrease in total TGF-β levels after a 48-hour exposure in keloid fibroblasts culture. Employing about 8 μM, TGF-β was reduced from about 200 pg/ml to about 50 pg/ml, representing a percent reduction of about 75%.

### EXAMPLES

Example #1 is an embodiment in which the topical scar treatment composition is employed in women who have suffered breast cancer and have undergone a mastectomy. Generally, anti-estrogen drugs, such as tamoxifen citrate, are used to treat breast cancer patients, and are also needed after a mastectomy. Thus, in this embodiment, because a mastectomy may lead to excessive scarring, topical scar treatment composition may be applied after a mastectomy, assisting on post-cancer treatment and preventing excessive scarring.

Example #2 is an embodiment in which the topical scar treatment composition is utilized after the surgical removal of abnormal scars, which is normally performed in complicated cases of excessive scarring after 2 years. Because surgical removal of abnormal scars may commonly lead to new excessive scarring, topical scar treatment composition may assist on preventing new or old scars from appearing.

While various aspects and embodiments have been disclosed, other aspects and embodiments are contemplated. The various aspects and embodiments disclosed are for purposes of illustration and are not intended to be limiting, with the true scope and spirit being indicated by the following claims.

What is claimed is:

1. A method of treating or reducing the risk of excessive scarring, comprising locally cutaneously administering tamoxifen citrate to a site of the excessive scarring or of a wound or incision at risk for developing the excessive scarring.

2. A method according to claim 1, further comprising a pharmaceutically acceptable carrier wherein the tamoxifen citrate is present in the pharmaceutically acceptable carrier at about 0.01% to about 1% by weight.

3. A method according to claim 2, further wherein the pharmaceutically acceptable carrier is anhydrous silicone.

4. A method according to claim 1, wherein said site comprises a keloid scar.

5. A method according to claim 1, wherein said site comprises a wound or incision at risk for developing a keloid scar.

6. A method according to claim 1, wherein said site comprises a hypertrophic scar.

7. A method according to claim 1, wherein said site comprises a wound or incision at risk for developing a hypertrophic scar.

8. A method according to claim 1, wherein said tamoxifen citrate causes a reduction of TGF-β.

9. A method according to claim 1, wherein said tamoxifen citrate is formulated in a vehicle comprising a penetration enhancer.

10. A method according to claim 9, wherein said penetration enhancer comprises an anhydrous silicone base.

11. A method according to claim 9, wherein said penetration enhancer comprises an Amazonian oil.

12. A method according to claim 11, wherein said Amazonian oil comprises one selected from the group comprising pracaxi oil, seje oil, and combinations thereof.

13. A method according to claim 11, wherein said Amazonian oil is about 0.5% to about 15% by weight of the penetration enhancer.

14. A method according to claim 11, wherein said Amazonian oil is about 1% by weight of the penetration enhancer.

15. A method according to claim 1, wherein the excessive scarring comprises mastectomy scarring.

16. A method according to claim 1, wherein the site comprises a site of removal of an abnormal scar.
